(19) United States
(12) Patent Application Publication
(10) Pub. No.: US 2019/0254949 A1
(43) Pub. Date: Aug. 22, 2019

(54) PRESSURE-SENSITIVE ADHESIVE NAIL COMPOSITIONS

(71) Applicant: L'OREAL, PARIS (FR)

(72) Inventors: Tsang-Min HUANG, Scotch Plains, NJ (US); Chunhua LI, Hilsborough, NJ (US); Hy Si BUI, Piscataway, NJ (US); Siliu TAN, Westfield, NJ (US)

(73) Assignee: L'OREAL, PARIS (FR)

(21) Appl. No.: 15/900,035

(22) Filed: Feb. 20, 2018

(51) Int. Cl. A61K 8/81; A61Q 3/02; A61K 8/86; A61K 8/04

(52) U.S. Cl. CPC A61K 8/817; A61Q 3/02; A61K 8/86; A61K 2800/95; A61K 8/042; A61K 2800/81; A61K 8/8135

(57) ABSTRACT

The invention relates to anhydrous nail compositions comprising interpolymer complexing compounds and a polar solvent. It further relates to methods of using such compositions.

### FIELD OF THE INVENTION

The present invention relates to nail compositions, and in particular, nail compositions that form a pressure-sensitive adhesive layer on the nail.

### DISCUSSION OF THE BACKGROUND

UV gel compositions are compositions applied to the nails to provide very durable color and shine. UV gel compositions typically consist of a layer of basecoat for adhesion on the nails, one or more color coats to enhance the color, and a layer of topcoat for shine. Each coating needs to be cured with a UV Lamp or UV LED. A UV gel composition set is thus a system that typically contains base coat, color coat and top coat layers. The UV gel composition set's adhesion on the nail and the cohesion among the layers is often so strong that it is difficult to remove such composition sets from nails. To remove such UV gel products from nails, it is usually required to soak nails with harsh solvent such as acetone for 20 minutes or more, followed by scraping the product off the nail. Frequent and/or prolonged use of such solvents in this manner and associated scraping can damage nails such as, for example, by making them dry and brittle. At the same time, the removal process is time-consuming.

It would be desirable to possess a UV gel product which has one or more of the following properties (preferably, all of the following properties): good water-resistance, good wear and/or good adhesion, without prolonging the amount of time needed for removal from nails.

There remains a need for UV gel compositions which are safe and adhere well to nails, and which have some or all of the desired properties discussed above.

### SUMMARY OF THE INVENTION

According to a first aspect of the invention, a nail composition is provided. The nail composition includes from about 20% by weight to about 90% by weight of a polar solvent. The nail composition further includes at least about 10% by weight of interpolymer complexing compounds. The interpolymer complexing compounds include a water-soluble polymer comprising a plurality of carbonyl groups and a water soluble compound comprising a plurality of hydroxyl groups. The nail composition further includes from about 1% to about 30% of a water insoluble compound selected from a group consisting of water-insoluble polymer, a photocrosslinkable compound and combinations thereof. The nail composition is anhydrous.

According to a second aspect of the invention, a method of making up nails is provided. The method includes the steps of applying to nails an anhydrous composition that includes a polar solvent. The nail composition further includes interpolymer complexing compounds. The interpolymer complexing compounds include a water-soluble polymer and a water soluble compound. The method further includes drying the anhydrous composition, and thereby forming a pressure sensitive adhesive film on the nail based on a complex formed via the association between the first water-soluble polymer and the second water soluble polymer.

It is to be understood that both the foregoing general description and the following detailed description are exemplary and explanatory only, and are not restrictive of the invention.

### DESCRIPTION OF THE FIGURES

FIG. 1 depicts a plot of peel strength of compositions consistent with embodiments of the invention as well as comparative compositions.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9%-11% and “about 2%” means 1.8%-2.2%).

“A” or “an” as used herein means “at least one.”

As used herein, all ranges provided are meant to include including all combinations of such ranges and combination of subranges between the given ranges. Thus, a range from 1-5, includes specifically 1, 2, 3, 4 and 5, as well as subranges such as and 2-5, 3-5, 2-3, 2-4, 1-4, etc.

“Adhesion” as used herein, refers to chemical and/or physical bonding between a coating and a substrate. Good adhesion between nail polish and nail surface should translate to good wear properties on consumers.

“Adhesive agent” or “adhesive” means a polymer that improves chemical and/or physical bonding between a coating and a substrate. In this invention, the adhesive agent improves bonding between compositions and the nail surface or other compositions.

“Removal” or “Easy removal” means the composition may be substantially removed with acetone or other organic solvents not limited to butyl acetate, isopropyl alcohol, ethanol, ethyl acetate, methyl acetate, methyl ethyl ketone, and mixtures thereof, followed by scraping of the composition from the nail. In certain notable embodiments, the removal may be performed by peeling from the nails such as after soaking the nails in an aqueous composition.

“Film former”, “film-forming polymer” or “film forming agent” or “co-film former” as used herein means a polymer or resin that leaves a film on the substrate to which it is applied, for example, after a solvent accompanying the film former has evaporated, absorbed into and/or dissipated on the substrate.

“Free” or “devoid” of as it is used herein means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of”” means that non-aqueous solvents are preferably omitted (that is 0% by weight), but can be present in the composition at an amount of less than about 0.25% by weight, typically less than about 0.1% by weight, typically less than about 0.05% by weight, based on the total weight of the composition.

“Water free” or “free of water” or “anhydrous” herein means that water is preferably omitted (that is 0% by weight), but can be present in the composition at an amount of less than about 0.25% by weight, typically less than about 0.1% by weight, typically less than about 0.05% by weight, based on the total weight of the composition.

“Making up” as used herein means to provide decoration (for example, color) to the nail.

“Protecting” as used herein means to inhibit damage to the nail (for example, chipping) by providing a protective layer on the nail.

“Nails”, “fingernail or “toenail” refers to a human keratinous substrate on a finger or toe which can be treated (decorated) with a single or multiple nail cosmetic compositions.

“Nail treatment system” or “nail set” means multiple compositions formed on the surface of nails.

“Nail composition” or “lacquer” or “nail polish” or “nail enamel” refers to nail enamel usable as a basecoat, color coat, top coat, clear coat and protective coat applied on nails separately and/or as a combined application of the above.

“Organic solvent” refers to solvents that atomically include hydrogen and carbon and are generally low molecular weight (in certain embodiments less than about 1000 daltons) and are capable of dissolving various ingredients in a nail composition. These may be volatile at room temperature.

“Substituted” in a chemical sense, as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Gloss” in compositions as used herein refers to compositions having with an average gloss, measured at 20°, of greater than or equal to 35, for example 40, preferably 45, 55, 60 or 65, including all ranges and subranges therebetween such as 35-65, 40-65, etc., and/or an average gloss, measured at 60°, of greater than or equal to 65, 70, 75 or 80, including all ranges and subranges therebetween such as 65-80, 65-75, etc.

The term “average gloss” denotes the gloss as it can be measured using a gloss meter, for example by spreading a layer of the composition to be tested, between 50 μm and 150 μm in thickness, on a white Leneta contrast card using an automatic spreader. The deposit is cured under UV-LED lamp for 1 min. The residual tacky layer is wiped off with lint free cotton saturated in alcohol solvent, and then the gloss is measured at 20° using a Byk Gardner gloss meter of reference microTRl-GLOSS. This measurement is repeated at least three times, and the average gloss in GU (gloss units) is the average of the at least three measurements carried out.

The average gloss at 60° is measured in a similar manner, the measurement being carried out at 60° rather than 20°.

“On a solids basis,” as used herein, means considering only components (e.g., in a composition) that are solid at room temperature and ignoring portions of the composition that are liquid, e.g., water and other volatile solvents.

“Water resistance” as used herein, means resistance of a material (substance) to the penetration of water, which may cause degradation of that material. The method implemented if assessment of this invention is further disclosed

The compositions and methods of the present invention can comprise, consist of, or consist essentially of the essential elements and limitations of the invention described herein, as well as any additional or optional ingredients, components, or limitations described herein or otherwise useful.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

Basecoat

In accordance with the invention, the inventors have found that certain compositions can be advantageously used as basecoat compositions to provide a nail set that has the high durability and shine expected of a UV curable system, yet is easily removable.

Interpolymer Complexing Compounds (ICC)

According to certain embodiments of the invention a nail composition useful, for example, as a basecoat composition includes at least one water-soluble polymer and a at least one water-soluble compound dissolved in a polar solvent system. The water-soluble polymer and water-soluble compound are components of, and are collectively interpolymer complexing compounds (ICC). By “components of interpolymer complexing compounds”, it is meant that the water-soluble polymer and water-soluble compound are capable of associating with one another, especially as the polar solvent system is evaporated or otherwise drawn from the polymer(s).

The association of the components of the ICC allows for the forming of a cohesive film upon evaporation/dissipation of the polar solvent system (e.g., after the composition is coated onto a nail surface). Accordingly, upon coating the nail and the drying/removal of the solvent, a pressure-sensitive adhesive film may be formed on the nail surface.

In certain embodiments of the invention, the water-soluble compound that is capable of associating with the water-soluble polymer is a second water-soluble polymer. In certain other embodiments, the water-soluble compound that is capable of associating with the water-soluble polymer is a non-polymeric water-soluble compound.

The association of the water-soluble polymers (or association of the water-soluble polymer with the non-polymeric water-soluble compound) may be by, for example, hydrogen bonding. The resulting complex may therefore have high cohesive strength as compared with a film formed from conventional water-soluble polymers.

The water-soluble polymer and water-soluble compound each have sufficient polarity to them to render them soluble in the polar solvent system. Furthermore, according to certain embodiments, one component of the ICC has a plurality of electron donating groups (e.g, carbonyl groups) and the second component of the ICC has a plurality of proton donor groups (e,g, hydroxyl groups). Without wishing to be bound by theory it is believed that the one component of the ICC can serve as a reversible cross-linker for the other, thereby providing high cohesive strength. The combination of the sufficient cohesion, elasticity and water-susceptibility of the resulting film facilitates ease of removability of the nail set.

According to certain embodiments, one component of the ICC is a water-soluble polymer such as a polyvinyl amide, such as those selected from a cyclic amide such as polyvinylpyrrolidone and a polyvinylcaprolactam. Specific examples of suitable first water-soluble polymers include LUVIKSOL PLUS, commercially available from BASF of Ludwigshafen, Germany; and PVP K-60 (or Plasdone® K-60), PVPK-90 (or Plasdone® K-90), or PVP K-120 (or Plasdone® K-120), each commercially available from Ashland, Inc of Kovington, Ky.

According to certain embodiments, the second component of the ICC is a (second) water-soluble polymer or non-polymeric water-soluble compound such as a polyhydric alcohol such as, for example a polyoxyalkylene, an ethylene oxide/propylene oxide copolymer, and the like. In certain notable embodiments, the second water-soluble polymer is selected from polyoxyalkylenes such as polyethylene oxide (i.e., polyethylene glycol). According to certain other notable embodiments, the second water-soluble polymer is a polyethylene glycol having about 5 to 8 ethylene oxide repeat units. Specific examples of suitable polyethylene glycols include PLURACARE E, available from BASF of Ludwigshafen, Germany; and POGOL 400 available from Huntsman Corporation of The Woodlands, Tex.

Suitable non-polymeric water-soluble compounds useful to include in the ICC include those containing a plurality of hydroxyl groups such as polyhydric alcohols such as glycerin, diethylene glycol, triethylene glycol.

The components of the ICC may be present in the composition of the invention in a total (combined) concentration that is at least about 10%, such as at least about 30%, such as from about 10%, 15% 20%, 25%, or 30% to about 50%, 60% or 90% by weight, including all ranges and subranges there between, all weights being based on the total weight of the composition.

Furthermore, according to certain embodiments of the invention, the ICC may comprise at least about 65%, such as at least about 70%, such at least about 80% of the nail composition on a solids basis. In certain embodiments, the water-soluble polymer portion may comprise about 70% to about 95% of the nail composition on a solids basis.

Polar Solvent System

The interpolymer complexing compounds are dissolved in a polar solvent system. The polar solvent system may include, consist of, or consist-essentially of C2-C5 alcohols, water, blends of C2-C5 alcohols and water, chlorinated solvents, and nitroparrafins. In a notable embodiment, the polar solvent system includes, consists of or consists essentially of C2-C5 alcohols, water, or blends of C2-C5 alcohols and water. In certain other notable embodiments the polar solvent system includes, consists of or consists essentially of C2-C5 alcohols, such as ethanol. Accordingly, in certain embodiments of the invention, compositions are anhydrous. The polar solvent system may be present in a concentration from about 10%, 20%, 30%, or 40% to about 60%, 70%, 80% or 90% including all combinations of such ranges.

Water-Insoluble Compound

Aside from the solvent system and the at least two water-soluble polymers, the topcoat composition also includes a water-insoluble compound. The water-insoluble compound helps provide a moderate degree of water resistance to the basecoat film and to further enhance the peelability of the nail set. To provide sufficient water-resistance, the inventive basecoat composition may include from about 2%, 3% or 4% to about 10%, 12% of 15%, including all combinations of such ranges, of the water-insoluble compound.

Preferably, the water-soluble polymers and water-insoluble compound are present in the nail compositions of the present invention in a total water-soluble polymer to water-insoluble compound ratio of from about to about 1:1 to about 20:1, such as from about 1.5:1 to about 15:1; such as from about 2:1 to about 14:1; such as from about 5:1 to about 10:1.

In one embodiment, the water-insoluble compound is a water-insoluble polymer. Suitable water-insoluble polymers include those that are compatible with the solvent system (e.g., soluble in the solvent system), compatible with the first water-soluble polymer and the second water-soluble polymer, and having no or very limited water solubility. Polyesters, polyurethanes, epoxies, acrylics and vinyl polymers are all suitable for use as the water-insoluble polymer. One suitable polyester is a reaction product of adipic acid/neopentyl glycol/trimetallic anhydride, commercially available as UN IPLEX 670 P from Unitex Chemical (Dayton Superior) of Greensboro, N.C.

Furthermore, the water-soluble polymer may be a polymer that is hydrophobically modified to render it insoluble (or more insoluble than it would otherwise be) in water. Suitable hydrophobically-modified polymers include hydrophobically modified acrylic polymers and hydrophobically modified vinyl polymers. One suitable hydrophobically-modified vinyl polymer is a copolymer of vinyl acetate/croatonates/vinyl decanoate, commercially available as RESYN 28-2930 from AkzoNobel of Chicago, Ill.

In another embodiment, the water-insoluble compound is photocrosslinkable compound, such as a UV-crosslinkable compound. Suitable UV-crosslinkable compounds include those that are ethylenically unsaturated. The term “photocrosslinkable compound” refers to an organic compound suitable for crosslinking under the action of radiation such as UV, resulting in a crosslinked polymer network.

The photocrosslinkable compounds are preferably present at a total concentration greater than or equal to 0.5% by weight, in relation to the total weight of the composition, advantageously ranging from about 0.5% to about 90%, preferably from about 1% to about 30%, more preferably from about 1% to about 20%, advantageously from about 2% to about 15% by weight in relation to the total weight of the composition.

According to certain embodiments, the photocrosslinkable compound is at least one photocrosslinkable urethane (meth)acrylate compound. The term “urethane (meth)acrylate compound” refers to any compound comprising at least one urethane function —O—C(O)—NH—, also known as a carbamate, and at least one (meth)acrylate function according to the formula

H₂C═C(R)—C(O)—O—

where R═H or CH₃.

The “urethane” function is also referred to as a “carbamate” function. The urethane (meth)acrylate compound may be chosen from the group consisting of urethane poly(meth)acrylate compounds. According to the present invention, the term “poly(meth)acrylate compound” refers to a (meth)acrylate compound comprising a plurality of (meth)acrylate functions.

In this way, the term “poly(meth)acrylate compound” may refer to a compound comprising at least two methacrylate functions, or at least two acrylate functions, or at least one methacrylate function and at least one acrylate function.

As urethane (meth)acrylate compounds, particular mention may be made of urethane dimethacrylate compounds.

The term “urethane dimethacrylate compound” refers to any compound comprising at least one urethane function —O—C(O)—NH—, and two methacrylate functions according to the formula

H₂C═C(CH₃)—C(O)—O—.

The term “polyurethane group” refers to a group obtained from polymerizing a mixture of monomers comprising isocyanate functions and monomers.

Particularly preferred urethane (meth)acrylate compounds are those commercially available from Esstech, Inc of Essington, Pa. under the name Exothane such as, for example, Exothane 8, Exothane 9, Exothane 10, Exothane 24, Exothane 26, Exothane 32, Exothane 108, and Exothane 126. The Exothane compounds are elastomers having high conversion values properties leading to improved adhesion. For example, preferred compounds have conversion values of 80% or greater, preferably 85% or greater, preferably 90% or greater, including all ranges and subranges therebetween such as, for example, 83% to 99%, 85% to 99%, 90% to 99%, etc. Further, such compounds preferably have high viscosity, preferably between 8,500 cPs and 1,000,000, preferably between 10,000 cPs and 900,000 cPs, and preferably between 20,000 and 850,000 cPs at 25° C., including all ranges and subranges therebetween. Further, such compounds preferably have low shrinkage stress (MPa), preferably 1.0 or less, preferably 0.5 or less, preferably 0.3 or less, preferably 0.2 or less, including all ranges and subranges therebetween. Further, such compounds preferably have high elongation properties, preferably at least 10%, preferably at least 20%, preferably at least 30%, preferably at least 50%, and as high as 100% or higher, including all ranges and subranges therebetween such as, for example, 10% to 100%, 20% to 100%, etc. Further, such compounds preferably have low tensile strength (N/mm²), preferably less than 30 N/mm², preferably less than 25 N/mm², and preferably less than 20 N/mm².

(Meth)acrylate Monomer (Ethylenically unsaturated monomer)

According to preferred embodiments, the photocrosslinkable compound is at least one photocrosslinkable (meth)acrylate monomer. (Meth)acrylate monomer refers to a compound comprising a single (meth)acrylate function according to the formula H₂C═C(R)—C(O)—O—, where R═H or CH₃ capable of reacting with other molecules. In various embodiments, the at least one (meth)acrylate monomer may have a molecular weight ranging from 100 to about 300, for example, from about 120 to about 250.

In various embodiments, the at least one (meth)acrylate monomer may be chosen from compounds of general formula (I):

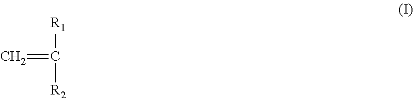

wherein:

R₁ is chosen from hydrogen and C₁-C₃₀ alkyl radicals and R₂ is chosen from —COOM radicals, wherein M is chosen from C₁-C₃₀ straight or branched chain alkyl radicals optionally substituted with at least one hydroxyl group or heterocycle, and from polyalkyleneoxy groups comprising preferably from 2 to 4 units, and from aromatic, alicyclic, and bicyclic rings optionally substituted with at least one substituent chosen from C₁-C₃₀ straight or branched chain alkyl radicals which may be substituted with at least one hydroxyl group. In another embodiment, the at least one (meth)acrylate monomer may be chosen from monomers of formula (I), wherein R₁ is chosen from hydrogen and CH₃, and R₂ is chosen from —COOM radicals, wherein M is chosen from C₁-C₁₀ straight or branched chain alkyl radicals optionally substituted with at least one hydroxyl group or heterocycle, and from aromatic, alicyclic, and bicyclic rings optionally substituted with at least one substituent chosen from C₁-C₃₀ straight or branched chain alkyl radicals which may be substituted with at least one hydroxyl group.

For example, the (meth)acrylate monomer may be chosen from (meth)acrylate monomers, such as methyl (meth)acrylate (MMA), ethyl (meth)acrylate (EMA), butyl (meth)acrylate (BMA), and polyethylene monomethacrylate such as diethylene glycol monomethacrylate, polypropylene glycol monomethacrylate such as dipropylene glycol monomethacrylate, and isobornyl (meth)acrylate, and tetrahydrofurfuryl (meth)acrylate (THFMA), and hydroxyalkyl (meth)acrylate monomers, such as hydroxypropyl methacrylate (HPMA), hydroxyethyl (meth)acrylate (HEMA), and butoxyethyl (meth)acrylate (BEMA).

Particularly useful for this invention is tetrahydrofurfuryl methacrylate (THFMA) available from Esstech, Inc. (X-958-7466).

Photoinitiator

For embodiments in which the water-insoluble compound is photocrosslinkable compound, the compositon may further include at least one photoinitiator. The photoinitiators suitable for use include those described, for example in “Les photoinitiateurs dans la reticulation des rev tements”, G. Li Bassi, Double Liaison—Chimie des Peintures, No. 361, November 1985, p. 34-41; “Applications industrielles de la polymerisation photoinduite”, Henri Strub, L′Actualite Chimique, February 2000, p. 5-13; and “Photopolymeres: considerations theoriques et reaction de prise”, Marc, J. M. Abadie, Double Liaison—Chimie des Peintures, No. 435-436, 1992, p. 28-34.

Suitable photoinitiators include, but are not limited to, alpha-hydroxyketones, marketed for example under the names DAROCUR® 1173 and 4265, IRGACURE® 184, 2959, and 500 by BASF, and ADDITOL® CPK by CYTEC, alpha.-aminoketones, marketed for example under the names IRGACURE® 907 and 369 by BASF, aromatic ketones marketed for example under the name ESACURE® TZT by LAMBERTI, thioxanthones marketed for example under the name ESACURE® ITX by LAMBERTI, and quinones (these aromatic ketones generally require the presence of a hydrogen donor compound such as tertiary amines and particularly alkanolamines—mention may particularly be made of the tertiary amine ESACURE® EDB marketed by LAMBERTI), alpha-dicarbonyl derivatives of which the most common is benzyl dimethyl ketal marketed under the name IRGACURE® 651 by BASF, and acylphosphine oxides, such as for example bis-acylphosphine oxides (BAPO) marketed for example under the names IRGACURE® 819, 1700, and 1800, DAROCUR® 4265, LUCIRIN® TPO, and LUCIRIN® TPO-L by BASF or PI-TPO-L from Esstech. Preferably, the photoinitiator is selected from the group consisting of alpha-hydroxyketones, alpha-aminoketones, aromatic ketones preferably associated with a hydrogen donor compound, aromatic alpha-diketones, acylphosphine oxides, and mixtures thereof.

Preferably, the at least one photoinitiator is present in the nail composition of the present invention in amounts of active material generally ranging from about 0.1% to about 10%, preferably from about 0.25% to about 7%, and more preferably from about 0.25% to about 5%, by weight, based on the total weight of the cosmetic composition, including all ranges and subranges in between.

In certain embodiments, the combined concentration of ethylenically unsaturated polymerizable compound and UV-photoinititiator is at least about 10% by weight of on a solids basis.

The topcoat may also include other additives or auxiliaries may be chosen from various functional ingredients such as gelling agents, thickeners, preservatives, fragrances, oils, surfactants, antioxidants, agents for combating free radicals, wetting agents, dispersing agents, antifoaming agents, neutralizing agents, stabilizing agents, active principles chosen from essential oils, UV screening agents, moisturizing agents, vitamins, proteins, ceramides, plant extracts, fibers, and the like, and their mixtures.

Color Coat

The color coats useful in the present invention may be any commercially available or otherwise suitable color coat, designed to impart color or optical effects to the nail.

Accordingly the color coat will include at least one colorant. Any colorant typically found in nail polish compositions can be used. Suitable colorants include, but are not limited to, lipophilic dyes, pigments, pearlescent agents, glitter, and their mixtures.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Preferably, if present, the at least one colorant is present in the nail composition of the present invention in amounts of active material generally ranging from about 0.1% to about 10%, preferably from about 0.25% to about 7%, and more preferably from about 0.5% to about 3.5%, by weight, based on the total weight of the cosmetic composition, including all ranges and subranges in between.

The color coat will also have ingredients typical of color coat compositions (e,g, film-formers, solvents, preservatives, and the like). In certain preferred embodiments, the color coat will include a photoinitiator and a photocrosslinkable compound, as described above for the topcoat.

Other additives or auxiliaries may be chosen from various functional ingredients such as gelling agents, thickeners, preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

Topcoat

Other compositions may be useful in the present invention, such as topcoats to enhance shine and durability. Any of various topcoats may be used. The topcoat will also have ingredients typical of top coat nail compositions (e,g, film-formers, solvents, preservatives, and the like). In certain preferred embodiments, the color coat will include a photoinitiator and a photocrosslinkable compound, as described above for the topcoat. In certain embodiments, the topcoat, if used will have in certain preferred embodiments, a photoinitiator and a photocrosslinkable compound, as described above.

Nail Sets

According to certain embodiments of the invention, a nail set is provided wherein the nail set includes a basecoat that is formed from interpolymer complexing compounds described herein. While in certain embodiments, the basecoat includes the water-insoluble compound and in certain other embodiments, the water-insoluble compound is excluded. The nail set further includes at least one color coat formed thereon. An optional topcoat may be formed on the one or more color coats.

Methods

The present invention also relates to methods for making up and/or protecting the nails. The method includes applying to (e.g., uncoated) nails a (e.g. fluid) composition that includes an ICC. The ICC includes a water-soluble polymer and a water-soluble compound that is capable of associating with the water-soluble polymer. The composition further includes a water-insoluble compound and a polar solvent system. The composition is allowed to dry thereby forming a pressure-sensitive adhesive on the nail based on a complex formed via the association between the water-soluble polymer and the second water-soluble compound. This forms a durable, “cured,” water-insoluble, and, in certain embodiments, glossy film on the nail. In certain notable embodiments, this film serves as a basecoat upon which one or more additional films are coated.

In certain notable embodiments of methods of the present invention, the composition is allowed to air dry—unassisted by exposure to focused radiation.

In certain alternative embodiments, the composition is cured via exposure to focused visible or ultraviolet (UV) radiation. Before the photocrosslinking occurs but after application of the nail composition of the present invention, there may be a period for drying the deposited coated layer, the duration of which may vary from 10 seconds to 10 minutes, typically from 30 seconds to 3 minutes. The drying is generally performed in air and at ambient temperature.

Suitable radiation crosslinking of the photocrosslinkable compound has, for example, a wavelength ranging from 210 to 600 nm, preferably from 250 to 420 nm, preferably from 350 to 410 nm. In one preferred embodiment of the invention methods, a LED lamp or an UV lamp, preferably a mercury vapor lamp, optionally doped with further elements, such as gallium, suitable for modifying the emission spectrum of the light source, can be used. Of course, the exposure time of the deposited coat to radiation is dependent on various factors such as the chemical nature and content of the reactive compounds or the crosslinking density sought. Typically, satisfactory results can be obtained after an exposure time ranging from 10 seconds to 100 minutes, preferably from 30 seconds to 5 minutes.

According to certain embodiments of methods of the present invention, the coating described above serves as a basecoat upon which one or more additional coatings are applied thereon to form a complete nail set on the nail. For example, a pigmented color-coat composition may be applied to the dry/cured basecoat and an optional topcoat may be applied to the dry/cured color-coat. According to certain embodiments after applying the color-coat and/or the topcoat, the nail may be exposed to focused radiation to cure (or further cure) the coatings on the nail. According to certain other embodiments, the resulting nail set may be soaked for several minutes in an aqueous composition (e.g., one free of organic solvents, such as one consisting of or consisting essentially of water) for a period of time to permit the wearer to peel at least the dried/cured color-coat (or entire nail set) free from the wearer's nails.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example A—Compositions and Peel Testing for Nail Set Having Basecoat Composition with Pressure Sensitive Adhesive

A basecoat nail formulation was prepared to test whether a pressure sensitive adhesive formed from an ICC included in a nail composition could provide peelability from a nail substrate. The composition (Example 1) included a first water-soluble polymer, a second water-soluble polymer and ethanol. Specifically, the following ingredients were combined: 40% by weight of polyvinyl caprolactam (itself a 40% solution in ethanol); 16% of polyethylene glycol; and 44% denatured ethanol. No water-insoluble compound was included. As a comparison a commercially available UV-curable basecoat was provided as well (Example 2). Both of these compositions were separately coated to a thickness of 3 mils onto buffed VITRONAILS (artificial nails which mimic human nails, commercially available from IMS, Inc. of Portland, Me.). The composition of Example 1 was allowed to dry at room temperature for 3 minutes and the composition of Example 2 was placed under UV irradiation with a commercially available UV lamp for 30 seconds). A commercially available topcoat was coated on top of the basecoat of Example 1 and the basecoat of Example 2 to a thickness of 3 mils, followed by wiping with isopropanol to remove and tacky layer on the topcoats.

A Peel Test was performed using an Instron with peeling test fixture. Three mils of base coat composition was coated on a buffed and cleaned vitro nail. Depending on whether the base coat was air dried or UV curable, the base coat was dried at room temperature for 3 minutes or under UV irradiation for 30 seconds. Then, a 3-mil top coat was coated on top of the base coat, followed by 60-second UV irradiation and IPA wiping to remove any tacky surface layer. The sample was then tested after 24-hour dry at room temperature.

For certain compositions. a Tack Test was also performed. Tack of pressure sensitive adhesives is measured using a texture analyzer from Stable Micro Systems (Godalming, UK). Samples are prepared by coating 3-mil PSA on a pre-cleaned glass plate, and dried at room temperature for 24 hours. The probe geometry used in the tack measurement is spherical. The probe approaches the sample surface with constant speed, 1 mm/sec. Once the probe contacts the sample surface, a constant force, 250 g, is applied on the sample for 10 seconds, and then the probe move away from the sample at constant speed. The maximum force measured during the probe/sample disengagement is defined as tack.

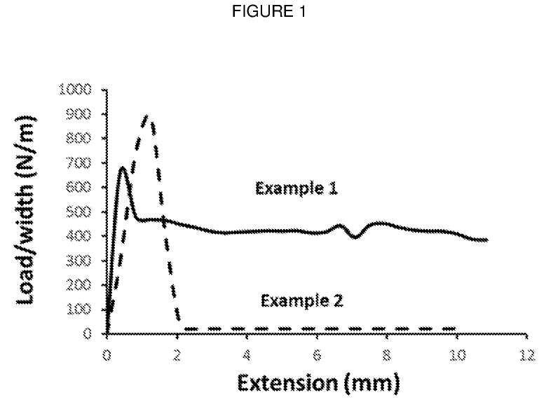

The results, shown in FIG. 1 show that the load associated with the ICC-based basecoat (Example 1) drops slightly as it peeled off, whereas the conventional basecoat (Example 2) breaks almost instantly and the load falls to zero.

### Example B—Compositions and Peel Testing for Nail Set Having Basecoat Compositions with Pressure Sensitive Adhesive That Includes Water-Insoluble Polymer

A second experiment was conducted in which water-insoluble polymer was included in the basecoat composition along with the ICC. Such formulations can be prepared by combining all raw materials, and then mixing in a high speed mixer. Compositions (weight percentage of ingredients) are shown in Table 1 below.

TABLE 1

| | Ex 3 | Ex 4 | Ex 5 | Ex 6 |
|---|---|---|---|---|
| Vinyl acetate/croatonates/vinyl decanoate | 0 | 5 | 10 | 24 |
| copolymer¹ [water-insoluble polymer] | | | | |
| Polyvinyl caprolactam (40% in ethanol [ICC] | 50 | 37.5 | 25 | 12.5 |
| Polyethylene glycol (8 EO) [ICC] | 20 | 20 | 20 | 20 |
| Denatured ethanol [polar solvent] | 30 | 37.5 | 45 | 52.5 |

¹vinyl acetate/croatonates/vinyl decanoate, commercially available as RESYN 28-2930 from AkzoNobel of Chicago, Illinois

The above compositions were tested for tack and peel strength. The results shown in Table 2 show that with the addition of water-insoluble polymer, Resyn-28-2930 at 5%, tack and peel strength drop only slightly and no residual film is left on the nail.

TABLE 2

| | Ex 3 | Ex 4 | Ex 5 | Ex 6 |
|---|---|---|---|---|
| Tack (grams) | 914 | 861 | 741 | 194 |
| Max Peel Strength | 451 | 441 | 93 | Not Tested |
| Peel Strength from Vitro Nail | 325 | 220 | 40 | Not Tested |
| Residual Film on Nail | Yes | no | no | Not Tested |

### Example C—Compositions and Peel Testing for Nail Set Having Basecoat Compositions with Pressure Sensitive Adhesive That Includes a Photocrosslinkable Water-Insoluble Compound

A third experiment was conducted in which water-insoluble photocrosslinkable compound was included in the basecoat composition along with ICC. Such formulations can be prepared by combining all raw materials, and then mixing in a high speed mixer. Compositions (weight percentage of ingredients) are shown in Table 3 below.

TABLE 3

| | Ex 7 | Ex 8 | Ex 9 | Ex 10 |
|---|---|---|---|---|
| Photocrosslinkable Compound/ | 0 | 12 | 24 | 44 |
| Photoinitiator Mixture² | | | | |
| Polyvinyl caprolactam (40% in ethanol) | 40 | 40 | 40 | 40 |
| Polyethylene glycol (8 EO) | 16 | 16 | 16 | 16 |
| Denatured ethanol | 44 | 32 | 20 | 0 |

²Mixture includes 4% photoinitiator, 71% isopropanol, 25% photocrosslinkable compounds.

The formulations were tested for tack, peel strength and observed for residual film left on after removal. The results are shown in Table 4 below.

TABLE 4

| | Ex 7 | Ex 8 | Ex 9 | Ex 10 |
|---|---|---|---|---|
| Tack (grams) | 570 | 929 | Not Tested | 712 |
| Max Peel Strength | 664 | 518 | Not Tested | 412 |
| Peel Strength from Vitro Nail | 420 | 400 | Not Tested | 400 |
| Residual Film on Nail | Yes | Partial | Not Tested | No |

The results shown in Table 4 show that with the addition of photocrosslinkable compound/photoinitiator, peel strength drops only slightly and residual film left on the nail can be reduced or eliminated.

What is claimed is:

1. A composition, comprising from about 20% by weight to about 90% by weight of a polar solvent;
at least about 10% by weight of interpolymer complexing compounds, comprising:
a water-soluble polymer comprising a plurality of carbonyl groups; and
a water soluble compound comprising a plurality of hydroxyl groups; and
from about 1% to about 30% of a water insoluble compound selected from a group consisting of water-insoluble polymer, a photocrosslinkable compound and combinations thereof; wherein the nail composition is anhydrous.

2. The composition of claim 1 at least about 30% by weight of interpolymer complexing compounds.

3. The composition of claim 1, wherein the polar solvent is a C2-C5 alcohol.

4. The composition of claim 1, wherein the water-soluble compound is a water-soluble polymer comprising a plurality of hydroxyl groups.

5. The composition of claim 1, wherein the water-soluble polymer is selected from polyvinylpyrrolidone and polyvinylcaprolactam.

6. The composition of claim 1, wherein the water-soluble compound is a polyoxyalkylene.

7. The composition of claim 1, wherein the interpolymer complexing compounds comprise at least about 80% of the nail composition on a solids basis.

8. The composition of claim 1, wherein the water-soluble compound is a non-polymeric compound selected from polyhydric alcohols.

9. The composition of claim 1, wherein the water insoluble compound is a water-insoluble polymer.

10. The composition of claim 1, wherein the water-insoluble polymer is selected from a group consisting of polyesters, epoxies, urethanes, acrylics, hydrophobically-modified polymers, and combinations thereof.

11. The composition of claim 1, wherein the water-insoluble polymer is selected from polyesters, hydrophobically-modified polymers, and combinations thereof.

12. The composition of claim 9, wherein the composition comprises from about 2% by weight to about 15% by weight of the water-insoluble polymer.

13. The composition of claim 1, wherein the water insoluble compound comprises an ethylenically unsaturated polymerizable compound and a UV-photoinitiator.

14. The composition of claim 13, wherein the UV-photoinititiator is selected from a group consisting alpha-hydroxyketones, alpha-aminoketones, aromatic ketones preferably associated with a hydrogen donor compound, aromatic alpha-diketones, acylphosphine oxides.

15. The composition of claim 13, wherein the composition comprises a combined concentration of at least about 10% by weight of ethylenically unsaturated polymerizable compound and UV-photoinititiator on a solids basis.

16. A composition, comprising
from about 20% by weight to about 90% by weight of a C2-C5 alcohol;
at least about 10% by weight of interpolymer complexing compounds, comprising:
a water-soluble polyvinylamide; and
a water soluble compound comprising a plurality of hydroxyl groups; and
from about 1% to about 30% of a water insoluble compound selected from a group consisting of water-insoluble polymer, a photocrosslinkable compound and combinations thereof; wherein the nail composition is anhydrous.

17. A method of making up nails comprising the steps of:
(1) applying to nails an anhydrous composition, comprising:
an interpolymer complexing compound, comprising:
a first water-soluble polymer; and
a second water soluble compound capable of associating with the first water-soluble polymer; and
a polar solvent; and
(2) drying the anhydrous composition thereby forming a pressure sensitive adhesive film on the nail based on a complex formed via the association between the first water-soluble polymer and the second water soluble polymer.

18. The method of claim 17 wherein the drying of the anhydrous composition comprises air-drying the anhydrous composition that has been applied to the nails.

19. The method of claim 17 wherein the drying of the anhydrous composition comprises curing the anhydrous composition that has been applied to the nails with an LED-source of ultraviolet radiation.

20. The method of claim 17 further comprising the step of (3) applying a pigmented composition on the dried pressure-sensitive adhesive coating on the nails to form a nail set.

21. The method of claim 20 further comprising the step of (4) soaking the nail set on the nails in an aqueous composition thereby permitting the dried color coating to peel free from the nails.

22. The method of claim 21 wherein the aqueous composition is free of organic solvents.

23. The method of claim 17 wherein the anhydrous composition further comprises a water insoluble compound.
